(19) United States
(12) Patent Application Publication
(10) Pub. No.: US 2022/0002715 A1
(43) Pub. Date: Jan. 6, 2022

(54) COMPOSITIONS AND METHODS FOR SELECTING BIALLELIC GENE EDITING

(71) Applicant: Ming Li, Phoenix, AZ (US)

(72) Inventor: Ming Li, Phoenix, AZ (US)

(21) Appl. No.: 17/288,849

(22) PCT Filed: Oct. 25, 2019
(86) PCT No.: PCT/US2019/058155

§ 371 Date: Apr. 26, 2021

Related U.S. Application Data
(60) Provisional application No. 62/750,635, filed on Oct. 25, 2018.

(51) Int. Cl. C12N 15/11; C12N 9/22; C12N 5/0783; G01N 33/569; C12N 15/90

(52) U.S. Cl. CPC C12N 15/11; C12N 9/22; C12N 5/0636; G01N 33/56966; C12N 2320/10; C12N 2310/20; C12N 2800/80; C12N 2510/00; C12N 15/907

(57) ABSTRACT

Disclosed are methods comprising administering CRISPR technology to a population of cells, wherein the CRISPR technology comprises one or more constructs for expressing a Cas protein, sgRNA against a marker gene, and sgRNA against a target sequence; and performing FACS-based negative selection to establish an enriched cell population of negatively selected cells; wherein the negatively selected cells do not have a marker encoded by the marker gene and do have a mutation in the target sequence. Disclosed are nucleic acid sequences comprising three elements, wherein a first element comprises a nucleic acid sequence that encodes a Cas protein, a second element comprises a nucleic acid sequence that expresses a sgRNA against a cell-surface marker gene, and a third element comprising a nucleic acid sequence that expresses a sgRNA against a target sequence.

### CROSS REFERENCE TO RELATED APPLICATIONS

This application claims the benefit of U.S. Application No. 62/750,635, filed on Oct. 25, 2018, the content of which is hereby incorporated by reference herein in its entirety.

### BACKGROUND

Recent progresses in designer nucleases has greatly improved the efficacy of gene editing, especially with the advent of Clustered Regularly-Interspaced Short Palindromic Repeats (CRISPR) technology. The latter relies on short guide RNAs (sgRNAs), or guide RNAs (gRNAs), to position Cas9, a nuclease of various prokaryotic origins towards the targeted genomic loci. The work flow of CRISPR gene editing has been described in detail by Ran et al. (Ran et al., 2013). Briefly, gRNAs against genes-of-interest are designed and cloned into targeting vectors for expression; the constructs are then introduced along with Cas9 expressing plasmid into target cells. The effectiveness of any gRNAs can be verified by conducting Suryeror or ENGEN assay in pooled edited cells, which takes advantage of a nuclease specializing in cutting at mismatches in dsDNA (heteroduplex) as a result of indel mutations introduced by non-homologous end-joining (NHEJ) repair after gene editing. The edited cells are then separated into individual single cell colonies which are expanded, with their individual mutations determined by PCR cloning of the targeted loci and Sanger sequencing. The entire process usually last 4 weeks, with most of human hours spent in isolation of single cell colonies and genotyping them for desired mutations. The number of colonies to be screened is significant, especially if one desires double knockout of the target genes in diploid eukaryotic cells, as the majority of resulting colonies have only mutations to a single allele.

In this disclosure, Flow Assisted Mutation Enrichment (FAME) is described, aiming to address the known problems of gene editing and improve on both efficacy and efficiency simultaneously. Using a FACS based negative selection for cells with ablation of a surface marker, this procedure significantly enriches for cells with double knockout at the desired loci, which are co-targeted along with the said negative selection marker, and the FACS procedure in the work-flow also provides the desired automation for single cell colony isolation. FAME procedure has significantly increased the prevalence of indel mutations in the negatively selected cells, with close to 100% of single cell colonies being DKOs.

### BRIEF SUMMARY

Disclosed are methods comprising administering CRISPR technology to a population of cells, wherein the CRISPR technology comprises one or more constructs for expressing a Cas protein, sgRNA against a marker gene, and sgRNA against a target sequence; and performing FACS-based negative selection to establish an enriched cell population of negatively selected cells; wherein the negatively selected cells do not have a marker encoded by the marker gene and do have a mutation in the target sequence.

Disclosed are methods comprising administering CRISPR technology to a population of cells, wherein the CRISPR technology comprises one or more constructs for expressing a Cas protein, sgRNA against a marker gene, and sgRNA against a target sequence; and performing FACS-based negative selection to establish an enriched cell population of negatively selected cells; wherein the negatively selected cells do not have a marker encoded by the marker gene and do have a mutation in the target sequence, and further comprising, after the negative selection step, performing sequence analysis.

Disclosed are recombinant cell comprising one or more constructs for expressing Cas-9, sgRNA against a cell-surface marker gene, and sgRNA against a target sequence.

Disclosed are nucleic acid sequences comprising three elements, wherein a first element comprises a nucleic acid sequence that encodes a Cas protein, a second element comprises a nucleic acid sequence that expresses a sgRNA against a cell-surface marker gene, and a third element comprising a nucleic acid sequence that expresses a sgRNA against a target sequence.

Disclosed are constructs comprising a nucleic acid sequence comprising three elements, wherein a first element comprises a nucleic acid sequence that encodes Cas-9, a second element comprises a nucleic acid sequence that expresses a sgRNA against a cell-surface marker gene, and a third element comprising a nucleic acid sequence that expresses a sgRNA against a target sequence.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example of the efficacy of different sgRNAs against B2M in ablating cell surface MHC-I antigen. HEK293T cells were transfected with combination of pCMV-spCas9 and different sgRNAs against B2M (B2M1, C; B2M2, D, and B2M3, E), and stained with FITC-conjugated antibody against human HLA-A,B&C. 5 days post-transfection. A and B were negative (no antibody staining) and positive (wild-type HEK293T cells stained with antibody), respectively.

FIG. 2 shows a work flow for FAME. See text for detailed explanation

FIGS. 3A-3G show enrichment of PTEN DKO with FAME strategy. A. HEK293T cells were transfected with expression plasmids for spCas9, sgRNAs for B2M1 and PTEN. 5 days later, transfected cells were stained with FITC-conjugated antibody against human HLA-A,B&C, and sorted into negative and positive populations in both pool and single cell (1 cell/well in 96-well plate) format, and expanded. B. The pooled cells were amplified for genomic DNA extraction, followed by EMGEN assay. 4 samples were included, including wild-type HEK293T cells, MHC-I positive pool, unsorted pool, and MHC-I negative pool. For each sample, both undigested and digested PCR products were run for comparison and quantification. C-G. Chromatogram from Sanger sequencing of the region targeted by sgRNA, including PAM (GGG). C, a typical wild type colony from MHC-I positive population identified with PCR sequencing; D, a typical DKO colony from MHC-I negative population with homozygous insertions identified with PCR sequencing; E, a colony (#4) from MHC-I negative population with illegible chromatogram with mixed signals; F and G, Sanger sequencing of plasmids with cloned PCR products from the same MHC-I negative colony as in E (#4) identified the exact mutations in each allele.

FIGS. 4A-4AB show enrichment of MYC and ZMIZ1 mutations with FAME strategy. HEK293T cells were transfected with expression plasmids for spCas9, sgRNAs for B2M1 and MYC (A) and (B). 5 days later, transfected cells were stained with FITC-conjugated antibody against human HLA-A,B&C, and sorted into negative and positive pools. After expansion of the pools, genomic DNA was extracted, followed by ENGEN assays. For each target (A and B), 4 samples were included, including wild-type HEK293T cells, MHC-I positive pool, unsorted pool, and MHC-I negative pool. For each sample, both undigested and digested PCR products were run for comparison and quantification.

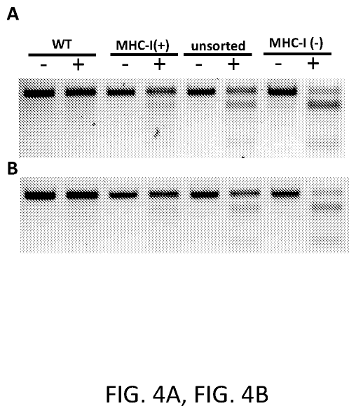

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a construct” includes a plurality of such constructs, reference to “the nucleic acid sequence” is a reference to one or more nucleic acid sequences and equivalents thereof known to those skilled in the art, and so forth.

As used herein, the term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element or regulatory element). The terms “plasmid” and “vector” can be used interchangeably, as a plasmid is a commonly used form of vector. Moreover, this disclosure is intended to include other vectors which serve equivalent functions.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

As used in the specification and in the claims, the term “comprising” can include the aspects “consisting of” and “consisting essentially of” “Comprising can also mean “including but not limited to.”

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods

Disclosed are methods of enriching the cell population comprising the desired mutations. In some aspects, the desired mutations are the double knockout mutations for both alleles of the desired target sequence. In some aspects, the presence of a marker gene being disrupted during gene editing can be used to also determine the presence of a target sequence being disrupted. Thus, in some aspects, the enriched cell population is lacking a marker encoded by a disrupted marker gene and is lacking a target protein encoded by a target sequence.

Disclosed are methods comprising administering CRISPR technology to a population of cells, wherein the CRISPR technology comprises one or more constructs for expressing a Cas protein, sgRNA against a marker gene, and sgRNA against a target sequence; and performing FACS-based negative selection to establish an enriched cell population of negatively selected cells; wherein the negatively selected cells do not have a marker encoded by the marker gene and do have a mutation in the target sequence.

Disclosed are methods comprising performing FACS-based negative selection on a population of cells that have undergone CRISPR gene editing to establish an enriched cell population of negatively selected cells; wherein the negatively selected cells do not comprise a marker encoded by a marker gene disrupted during gene editing and do comprise a mutation in a target sequence.

In some aspects, the CRISPR technology knocks out the marker gene and mutates the target sequence. In some aspects, the mutation of the target sequence is a double knock out in that both alleles of the target sequence are knocked out. In some aspects, the double knock out can be called a biallelic mutation.

In some aspects, the marker gene encodes a cell surface protein. A cell surface protein can be found on the cell membrane or cell surface of a cell and therefore can be detected by an extracellular antibody. In some aspects, the cell surface protein is any cell surface protein that is not essential for cell survival. In some aspects, the best cell surface proteins are those that are easily recognized by an extracellular antibody and are dispensable. In some aspects, the marker gene encodes β-2 microglobulin (B2M). In some aspects, any of the cluster of differentiation (CD) markers can be used as the marker.

In some aspects, the construct that expresses the sgRNA against a marker gene comprises the sequence CAGCCCAAGATAGTTAAGTGgttttagagctagaaatagc, ACAAAGTCACATGGTTCACAgttttagagctagaaatagc, or CTGAATCTTTGGAGTACCTGgttttagagctagaaatagc. In some aspects, the construct that expresses the sgRNA against a marker gene comprises the sequence CAGCCCAAGATAGTTAAGTG, ACAAAGTCACATGGTTCACA, or CTGAATCTTTGGAGTACCTG. Each of these sequences are B2M specific.

In some aspects, the marker gene encodes an intracellular protein. An intracellular protein can be found on the inside of the cell and therefore can be detected by an intracellular antibody. In some aspects, the intracellular protein is any intracellular protein that is not essential for cell survival.

In some aspects, the target sequence(s) can be selected from one or more of the nucleic acid sequences encoding PTEN, MYC and ZMIZ1. In some aspects, the construct that expresses the sgRNA against a target sequence comprises the sequence of ATGACCTAGCAACCTGACCAgttttagagctagaaatagc, CAGAGTAGTTATGGTAACTGgttttagagctagaaatagc, or TTGGTTACTCCCCAAACCGgttttagagctaggccaac. In some aspects, the construct that expresses the sgRNA against a target sequence comprises the sequence of ATGACCTAGCAACCTGACCA (PTEN specific sequence), CAGAGTAGTTATGGTAACTG (MYC specific sequence), or TTGGTTACTCCCCAAACCG (ZMIZ1 specific sequence).

In some aspects, the mutation is a biallelic indel mutation. In some aspects, the biallelic indel mutation is confirmed using sequence analysis.

Disclosed are methods comprising administering CRISPR technology to a population of cells, wherein the CRISPR technology comprises one or more constructs for expressing a Cas protein, sgRNA against a marker gene, and sgRNA against a target sequence; and performing FACS-based negative selection to establish an enriched cell population of negatively selected cells; wherein the negatively selected cells do not have a marker encoded by the marker gene and do have a mutation in the target sequence, and further comprising, after the negative selection step, performing sequence analysis. For example, the sequence analysis can be but is not limited to, Sanger sequencing.

Disclosed are methods comprising administering CRISPR technology to a population of cells, wherein the CRISPR technology comprises one or more constructs for expressing a Cas protein, sgRNA against a marker gene, and sgRNA against a target sequence; and performing FACS-based negative selection to establish an enriched cell population of negatively selected cells; wherein the negatively selected cells do not have a marker encoded by the marker gene and do have a mutation in the target sequence, further comprising, after the negative selection step, culturing the enriched cell population. In some aspects, the cells are cultured in order to increase the quantity so that sub-populations can be frozen. In some aspects, the cells are cultured long enough to grow enough cells to perform genomic isolation wherein further studies on the genome can be performed. In some aspects, the genome studies can include sequencing.

In some aspects, the population of cells are mammalian cells. In some aspects, the population of cells are a cell line. In some aspects, the population of cells are cultured primary cells. In some aspects, the population of cells can be, but are not limited to, T cells.

In some aspects, FACS-based negative selection comprises administering an antibody directed to the marker. In some aspects, the antibody can be an anti-MHC I antibody. In some aspects, the antibody can be an anti-B2M antibody.

The disclosed methods can enrich for cells with double knockout at the desired loci.

Disclosed are methods comprising selectively knocking out a gene in a cell, wherein the gene is autosomal and encodes for a cell surface marker; and screening the cells using FACS; wherein said FACS identifies cells that lack the cell surface marker.

In some aspects, the methods can further comprise introducing a fluorescent marker into the cell. In some aspects, a fluorescent marker can be, but is not limited to, green, red, yellow or blue fluorescent protein, mCherry, fluorescein, APC (allophycocyanin); FITC (fluorescein isothiocyanate); PE (phycoerythrin); PerCP (peridinin chlorophyll protein, Alexa Fluor.

In some aspects, the methods further comprise knocking out a gene of interest or target sequence.

In some aspects, the autosomal gene that encodes for a cell surface marker gene is B2M.

In some aspects, the selective knock out is performed using CRISPR. Thus, any of the known CRISPR techniques or those described herein can be used.

In some aspects, the FACS can be used to select MHC-1 negative cells. In some aspects, the MHC-I negative cells are cells wherein the B2M has been knocked out.

1. CRISPR Technology or CRISPR System

One or more constructs or vectors can be introduced into a cell (e.g., a host cell) to produce transcripts, proteins, peptides including fusion proteins and peptides, encoded by nucleic acids as described herein (e.g., clustered regularly interspersed short palindromic repeats (CRISPR) transcripts, proteins, enzymes, mutant forms thereof, fusion proteins thereof, etc.). Any of the constructs/vectors disclosed herein can be used in the current invention.

The vector or vector systems disclosed herein can comprise one or more vectors. Vectors can be designed for expression of CRISPR transcripts (e.g., nucleic acid transcripts, proteins, or enzymes) in prokaryotic or eukaryotic cells. CRISPR transcripts, for example, can be expressed in bacterial cells (e.g., Escherichia coli), insect cells (using baculovirus expression vectors), yeast cells, or mammalian cells. Alternatively, the recombinant expression vector can be transcribed and translated in vitro, for example, using T7 promoter regulatory sequences and T7 polymerase.

Generating constructs for the CRISPR/Cas9 system described herein can be a singleplex or multiplexed. The targets of the CRISPR/Cas9 system described herein can be multiplexed. In some aspects, the vectors can be singleplex vector or multiplex vectors. In some aspects, the singleplex or multiplex vectors can be repression or downregulation vectors or upregulation vectors or a combination thereof.

Vectors can be introduced in a prokaryote, amplified and then the amplified vector can be introduced into a eukaryotic cell. The vector can also be introduced in a prokaryote, amplified and serve as an intermediate vector to produce a vector that can be introduced into a eukaryotic cell (e.g., amplifying a plasmid as part of a viral vector packaging system). A prokaryote can be used to amplify copies of a vector and express one or more nucleic acids to provide a source of one or more proteins for delivery to a host cell or host organism. Expression of proteins in prokaryotes is often carried out in Escherichia coli with vectors containing constitutive or inducible promoters directing the expression of either fusion or non-fusion proteins.

In some aspects, the Cas protein, sgRNA against a marker gene, and sgRNA against a target sequence are expressed from different constructs. Thus, in some aspects, at least three different constructs can be used. In some aspects, the Cas protein, sgRNA against a marker gene, and sgRNA against a target sequence are expressed from the same construct. For example, a construct can comprise a nucleic acid sequence, wherein the nucleic acid sequence comprises three elements, wherein a first element comprises a nucleic acid sequence that encodes Cas-9, a second element comprises a nucleic acid sequence that expresses a sgRNA against a cell-surface marker gene, and a third element comprising a nucleic acid sequence that expresses a sgRNA against a target sequence.

In some aspects, the Cas9 can be a Streptococcus pyogenes Cas9 (SpCas9). The Streptococcus pyogenes Cas9

As used herein, “CRISPR system” and “CRISPR-Cas system” refers to transcripts and other elements involved in the expression of or directing the activity of CRISPR-associated (“Cas”) genes, including sequences encoding a Cas gene, a guide sequence (also referred to as a “spacer” in the context of an endogenous CRISPR system; e.g. guide RNA or gRNA), or other sequences and transcripts from a CRISPR locus. In some aspects, one or more elements of a CRISPR system is derived from a type I, type II, or type III CRISPR system. In some aspects, one or more elements of a CRISPR system are derived from a particular organism comprising an endogenous CRISPR system, such as Streptococcus pyogenes. Generally, a CRISPR system is characterized by elements that promote the formation of a CRISPR complex at the site of a target sequence (also referred to as a proto spacer in the context of an endogenous CRISPR system).

As used herein, the term “target sequence” refers to a sequence to which a guide sequence (e.g. gRNA/sgRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence can comprise any polynucleotide, such as DNA or RNA polynucleotides. In some aspects, a target sequence can be located in the nucleus or cytoplasm of a cell. In some aspects, the target sequence can be within an organelle of a eukaryotic cell (e.g., mitochondrion). A sequence or template that can be used for recombination into the targeted locus comprising the target sequences is referred to as an “editing template” or “editing polynucleotide” or “editing sequence.” In an aspect, the target sequence(s) can be selected from one or more of the nucleic acid sequences encoding PTEN, MYC and ZMIZ1. In an aspect, the target sequence(s) can be any oncogene or any gene in which inhibition or modulation of the gene activity would be beneficial for a subject. In some aspects, the term “target sequence” and “gene of interest” can be used interchangeably.

A guide sequence or single guide sequence (e.g. gRNA or sgRNA) can be any polynucleotide sequence having sufficient complementarity with a target polynucleotide sequence to hybridize with the target sequence and direct sequence-specific binding of a CRISPR-Cas system or CRISPR complex to the target sequence. In some aspects, the degree of complementarity between a guide sequence (e.g. gRNA) and its corresponding target sequence is about or more than about 50%, 60%, 70%, 75%, 80%, 85%, 90%, 95%, 99%, or more. In some aspects, a guide sequence is about more than about 5, 10, 15, 20, 25, 30, 35, 40, 45, 50 or more nucleotides in length or any number in between. gRNA and sgRNA can be used interchangeably.

The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). It is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). A skilled person will be able to identify further PAM sequences for use with a given CRISPR enzyme. In an aspect, the PAM comprises NGG (where N is any nucleotide, (G)uanine, (G)uanine).

Disclosed herein, are gRNA sequences. The disclosed gRNA sequences can be specific for one or more desired target sequences. In some aspects, the gRNA sequences can be specific to a marker gene. A marker gene can be any sequence that encodes a protein that is easily selectable, autosomal, can be targeted using FACS, and is dispensable for survival and important cellular functions. In some aspects, the gRNA sequence hybridizes with a target sequence of a DNA molecule or locus in a cell. In some aspects, the target sequences can be selected from one or more of the sequences listed in Table 1. In some aspects, the cell can be a mammalian or human cell.

In some aspects, the gRNA targets and hybridizes with the target sequence and directs the RNA-directed nuclease to the DNA locus. In some aspects, the CRISPR-Cas system and vectors disclosed herein comprise one or more gRNA sequences. In some aspects, the CRISPR-Cas system and vectors disclosed herein comprise 2, 3, 4 or more gRNA sequences. In some aspects, the CRISPR-Cas system and/or vector described herein comprises 4 gRNA sequences in a single system. In some aspects, the gRNA sequences disclosed herein can be used turn one or more genes on (p300core, tripartitie activator, VP64-p65-Rta (VPR)) or off (KRAB).

The compositions described herein can include a nucleic acid encoding a RNA-directed nuclease. The RNA-directed nuclease can be a CRISPR-associated endonuclease. In some aspects, the RNA-directed nuclease is a Cas9 nuclease or protein. In some aspects, the Cas9 nuclease or protein can have a sequence identical to the wild-type Streptococcus pyrogenes sequence. In some aspects, the Cas9 nuclease or protein can be a sequence for other species including, for example, other Streptococcus species, such as thermophilus; Psuedomona aeruginosa, Escherichia coli, or other sequenced bacteria genomes and archaea, or other prokaryotic microogranisms. In some aspects, the wild-type Streptococcus pyrogenes sequence can be modified. In some aspects, the nucleic acid sequence can be codon optimized for efficient expression in eukaryotic cells.

Disclosed herein, are CRISPR-Cas systems, referred to as CRISPRi (CRISPR interference), that utilizes a nuclease-dead version of Cas9 (dCas9). In some aspects, the dCas9 can be used to repress expression of one or more target sequences (e.g., tumor necrosis factor receptor (e.g., TNFR2), interleukin 1 receptor (e.g, IL1R2, IL6R), A-kinase anchor protein 5 (e.g., AKAP5, a glycoprotein (e.g., gp130) and transient receptor potential cation channel subfamily V member 1 (TRPV1)). Instead of inducing cleavage, dCas9 remains bound tightly to the DNA sequence, and when targeted inside an actively transcribed gene, inhibition of, for example, pol II progression through a steric hindrance mechanism can lead to efficient transcriptional repression. In some aspects, the dCas9 can be used to induce expression of one or more target sequences (e.g., PTEN, MYC).

In some aspects, the CRISPR system can be used in which the nucleus has been deactivated. Further, a KRAB, VPR or p300 core can be attached. In some aspects, the KRAB is attached to downregulate one or more genes in a cell. In some aspects, the p300core or VPR can be attached to upregulate one or more genes in a cell.

### C. Recombinant Cells

Disclosed are recombinant cells comprising one or more of the disclosed nucleic acid sequences or constructs.

Disclosed are recombinant cell comprising one or more constructs for expressing Cas-9, sgRNA against a cell-surface marker gene, and sgRNA against a target sequence.

In some aspects, the cell-surface marker gene encodes a cell-surface protein that is not essential for cell survival. In some aspects, the cell-surface marker gene encodes β-2 microglobulin. In some aspects, the construct that expresses the sgRNA against a cell-surface marker gene comprises the sequence CAGCCCAAGATAGTTAAGTGgttttagagctagaaatagc, ACAAAGTCACATGGTTCACAgttttagagctagaaatagc, or CTGAATCTTTGGAGTACCTGgttttagagctagaaatagc.

In some aspects, the target sequence is a nucleic acid sequence encoding PTEN, MYC or ZMIZ1. In some aspects, the construct that expresses the sgRNA against a target sequence comprises the sequence of ATGACCTAGCAACCTGACCAgttttagagctagaaatagc, CAGAGTAGTTATGGTAACTGgttttagagctagaaatagc, or TTGGTTACTCCCCAAACCGgttttagagctaggccaac.

In some aspects, the cell is a T cell. In some aspects, the cell is a mammalian cell.

In some aspects, the cells comprise constructs for expressing sgRNA against a cell-surface marker gene and sgRNA against a target sequence. In some aspects, the cells further comprise a nucleic acid sequence that encodes a Cas protein in their genome.

### D. Nucleic Acid Sequences

Disclosed are nucleic acid sequences comprising three elements, wherein a first element comprises a nucleic acid sequence that encodes a Cas protein, a second element comprises a nucleic acid sequence that expresses a sgRNA against a cell-surface marker gene, and a third element comprising a nucleic acid sequence that expresses a sgRNA against a target sequence.

Disclosed are nucleic acid sequences comprising at least two of three elements, wherein a first element comprises a nucleic acid sequence that encodes a Cas protein, a second element comprises a nucleic acid sequence that expresses a sgRNA against a cell-surface marker gene, and a third element comprising a nucleic acid sequence that expresses a sgRNA against a target sequence. In some aspects, a nucleic acid sequence comprises a nucleic acid sequence that expresses a sgRNA against a cell-surface marker gene and a nucleic acid sequence that expresses a sgRNA against a target sequence.

In some aspects, a Cas protein can be Cas-9.

In some aspects, the cell-surface marker gene encodes a cell surface protein. A cell surface protein can be found on the cell membrane or cell surface of a cell and therefore can be detected by an extracellular antibody. In some aspects, the cell surface protein is any cell surface protein that is not essential for cell survival. In some aspects, the best cell surface proteins are those that are easily recognized by an extracellular antibody and are dispensable. In some aspects, the marker gene encodes β-2 microglobulin (B2M). In some aspects, any of the cluster of differentiation (CD) markers can be used as the marker.

### E. Constructs

Disclosed are constructs comprising any one of the disclosed nucleic acid sequences. For example, disclosed are constructs comprising a nucleic acid sequence comprising three elements, wherein a first element comprises a nucleic acid sequence that encodes Cas-9, a second element comprises a nucleic acid sequence that expresses a sgRNA against a cell-surface marker gene, and a third element comprising a nucleic acid sequence that expresses a sgRNA against a target sequence. In some aspects, the first element, second element and third element are operably linked.

Disclosed are constructs comprising a nucleic acid sequence comprising a nucleic acid sequence that expresses a sgRNA against a cell-surface marker gene and a nucleic acid sequence that expresses a sgRNA against a target sequence. In some aspects, the nucleic acid sequence that expresses a sgRNA against a cell-surface marker gene and the nucleic acid sequence that expresses a sgRNA against a target sequence are operably linked.

In some aspects, the construct that expresses the sgRNA against a marker gene comprises the sequence CAGCCCAAGATAGTTAAGTGgttttagagctagaaatagc, ACAAAGTCACATGGTTCACAgttttagagctagaaatagc, or CTGAATCTTTGGAGTACCTGgttttagagctagaaatagc. In some aspects, the construct that expresses the sgRNA against a marker gene comprises the sequence CAGCCCAAGATAGTTAAGTG, ACAAAGTCACATGGTTCACA, or CTGAATCTTTGGAGTACCTG. Each of these sequences are B2M specific.

In some aspects, the construct that expresses the sgRNA against a target sequence comprises the sequence of ATGACCTAGCAACCTGACCAgttttagagctagaaatagc, CAGAGTAGTTATGGTAACTGgttttagagctagaaatagc, or TTGGTTACTCCCCAAACCGgttttagagctaggccaac. In some aspects, the construct that expresses the sgRNA against a target sequence comprises the sequence of ATGACCTAGCAACCTGACCA (PTEN specific sequence), CAGAGTAGTTATGGTAACTG (MYC specific sequence), or TTGGTTACTCCCCAAACCG (ZMIZ1 specific sequence).

In some aspects, the vector is a viral vector. Examples of viral vectors include, but are not limited to lentiviruses, adenoviral, and adeno-associated viruses. The type of vector can also be selected for targeting a specific cell type. In some aspects, vectors can also be an expression vector, for example, a yeast expression vector (e.g., Saccharomyces cerivisaie). In some aspects, the vector is capable of driving expression of one or more sequences in mammalian cells using a mammalian expression vector. Examples of mammalian expression vectors include but are not limited to pCDM8 and pMT2PC. In mammalian cells, regulatory elements control the expression of the vector. Examples of promoters are those derived from polyoma, adenovirus 2, cytomegalovirus, simian virus 40, and others disclosed herein and known in the art.

The vectors disclosed herein can comprise one or more promoters or regulatory elements or the like. In an aspect, a vector comprises one or more pol promoters, one or more pol promoters II, one or more poll III promoters, or combinations thereof. Examples of pol II promoters include, but are not limited to the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phospho glycerol kinase (PGK) promoter, and the EF1α promoter. In some aspects, pol II promoters can be engineered to confer tissue specific and inducible regulation of gRNAs. Examples of pol III promoters include, but are not limited to, U6 and H1 promoters. In an aspect, the promoter is U6. In an aspect, the promoter operably linked to the gRNA is a Pol III promoter, human u6, mouse U6, H1, or 7SK.

In some aspects, the compositions described herein (e.g., CRISPR-Cas systems, vectors) can comprise one or more promoters or regulatory elements. In the instance of two or more promoters or regulatory elements, said promoters or regulatory elements can be referred to as a first promoter, a second promoter and so on.

Generating constructs for the CRISPR/Cas9 system described herein can be a singleplex or multiplexed. The targets of the CRISPR/Cas9 system described herein can be multiplexed. In some aspects, the vectors can be singleplex vector or multiplex vectors. In some aspects, the singleplex or multiplex vectors can be repression or downregulation vectors or upregulation vectors or a combination thereof.

In some aspects, the regulatory element is operably linked to one or more elements of a CRISPR system to drive expression of the one or more elements of the CRISPR system. CRISPRs are a family of DNA loci that are generally specific to a particular species (e.g., bacterial species). The CRISPR locus comprises a distinct class of interspersed short sequence repeats (SSRs) that were identified in E. coli, and associated genes. The repeats can be short and occur in clusters that are regularly spaced by unique intervening sequences with a constant length.

In some aspects, the vector comprises a regulatory element operably linked to an enzyme-coding sequence encoding a CRISPR enzyme (e.g., a Cas protein). In some aspects, the CRISPR enzyme can be Cas9 and can be from Streptococcus pyogenes, Streptococcus thermophiles, or Treponema centicola. In some aspects, the Cas9 can be dCas9. In some aspects, the Cas9 protein can be codon optimized for expression in the cell.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed nucleic acid sequences, constructs, or cells. The kits also can contain reagents for culturing cells.

### Examples

1. Previous Arts

FACS positive selection of cells that have undergone biallelic mutagenesis based on site specific insertion of fluorescence markers by homology directed repair (HDR). This method relies on sequential targeting of each allele of the gene-of-interest and inserting expression cassettes for different fluorescent proteins at the mutation sites by way of HDR. This will allow cells with both alleles mutated to be positively selected with FACS. Although this has the advantage of near 100% success rate in selected cells, the work flow is too complex to be practical. For each gene being targeted, one needs to not only create the construct for sgRNA, but also repair templates with gene-specific arms and different fluorescent proteins. For cells that are difficult to transfect, such strategy will be nearly impossible (Wu et al., 2017).

Negative selection by co-targeting Hypoxanthine-guanine phosphoribosyltransferase (HPRT) has been used to enrich for DKO cells. HPRT is an enzyme in the rescue pathway of purine synthesis. It is not essential for cell survival; yet its deficiency in host cells protects against cytotoxic drug 6-thioguannine (6TG). This feature has been taken advantage of to enrich for DKOs in nuclease-modified cells when both gene-of-interest and HPRT are co-targeted. The results are variable, with the percentage of DKO under 5% in successful runs (Moriarity et al., 2014). The unsatisfactory results likely reflect the fact that HPRT is X-linked, therefore in any given sex, only one functional copy is in force. Therefore, 6TG resistant colonies result from modification of only a single allele, which is likely not adequate for selection of double knockouts. In addition, isolation of 6TG colonies requires manual labor and is time-consuming comparing to automated single cell isolation from FACS.

2. Rationale of Current Invention

To solve the above problems, a better strategy would combine the strengths of the above two methods, e.g., a FACS-based negative selection strategy. The success of this approach depends on an appropriate negative selection marker. An ideal marker needs to satisfy the following four conditions, i.e. 1) easily selectable; 2) autosomal thus biallelic, so that negative selection enriches for cells with highest efficiency for gene editing; 3) supporting automation of single cell isolation, such as with FACS; and importantly 4) dispensable for survival and important cellular functions.

A membrane protein beta2-microglobulin (B2M) could potentially satisfy all above conditions. B2M is a component of the type I Major Histocompatibility Complex (MHC-I), thus universally present in all tissue types. It can be readily detected with flow cytometry with antibodies against either B2M itself or the MHC-I complex. Cells with surface ablation of B2M can thus be negatively selected with FACS. B2M gene is autosomal with 2 alleles located on chromosome 15; complete ablation of surface B2M protein requires loss-of-function mutations in both alleles, thus requiring presence of a highly effective editing machinery in the targeted cells. This requirement will translate into more effective editing in other loci being targeted at the same time, allowing DKOs for the genes-of-interest to be enriched in the negatively sorted cell population.

Importantly, B2M and MHC-I are not essential for cell survival and largely dispensable, especially in ex vivo settings. Being an essential component of MHC-I, B2M is important for development and execution of cell-mediated immunity. Mice with B2M knockout are viable despite being immunodeficient for lack of CD8+ lymphocytes. Cells lacking B2M are hypoimmunogenic and protected from cell-mediated immunity as they could not be recognized by CD8+ T lymphocytes. This feature can be taken advantage of to generate off-shelf and hypoimmunogenic cell therapy products originating from allogenic or xenogenic donors, thus greatly reducing the cost of cell therapy. In this setting, ablation of B2M can serve two purposes at the same time, including enriching for desired therapeutic genetic modifications, and offering protection against rejection of implanted cells. In an earlier proof-of-principle experiment, it was reported that human embryonic stem cells (hESC) with the surface B2M ablated were able to develop into teratoma once implanted into immunocompetent mouse, thereby showcasing both the negligible functional consequence of B2M ablation and its usefulness in developing cell therapy (Wang, Quan, Yan, Morales, & Wetsel, 2015). Ablation of B2M has also been instrumental in producing hypoimmunogenic CAR-T cells with little Graft-versus-Host Disease (Ren et al., 2017).

3. Key Components of the Current Invention

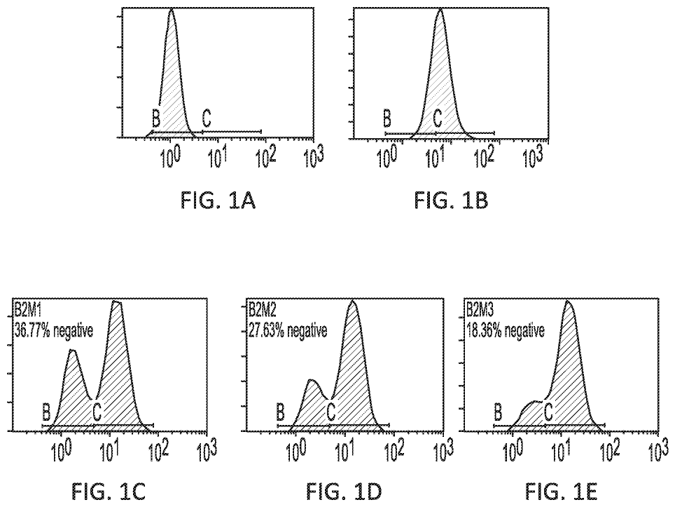

Developing sgRNA and Cas9 pairs for surface ablation of B2M. Three sgRNAs have been designed using cloud-based predicting software from Desktop genetics (London, UK) with predicted high targeting efficiency and low off-target editing (Table 1). The efficacy has been tested of all three with flow cytometry after transfecting them along with expression plasmid for spCas9. All three were able to produce significant percentage of cells with negative expression of MHC-I in the resulting cells, with highest ablation rate seen in sgB2M1 (FIG. 1). In the subsequent experiments, this gRNA was used to ensure adequate sensitivity of the experiments. The other less efficacious sgRNAs, however, can be useful in situations where higher selection pressure is needed.

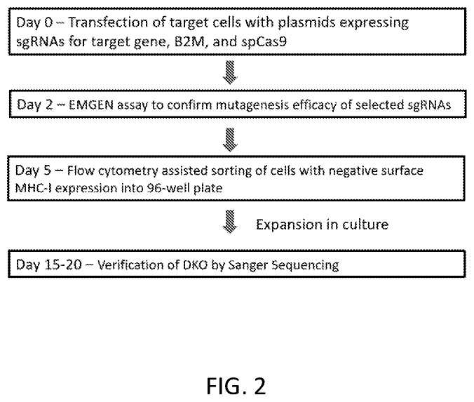

Enrichment of DKOs by FAME. The proposed work flow for FAME is illustrated in FIG. 2. On day 0, plasmids for expressing Cas9 and sgRNAs against B2M and gene-of-interest are transfected into target cells; 2 days after transfection, a fraction of the transfected cells were collected for genomic DNA extraction, followed by ENGEN assay, which recognizes and digests double-stranded DNA with mismatches (heteroduplex), to verify the efficacy of the sgRNAs in introducing indel mutations; on day 5, the transfected cells are stained with anti-MHC-I antibody and run under FACS to select for cells with surface MHC-I ablation, and the negative population are sorted into single cells in 96-well plates to expand into monoclonal colonies. The presence and nature of the mutations in each colony is then determined by PCR cloning and Sanger sequencing. The entire process should take around 2-3 weeks, dependent on the proliferation rate of the cells.

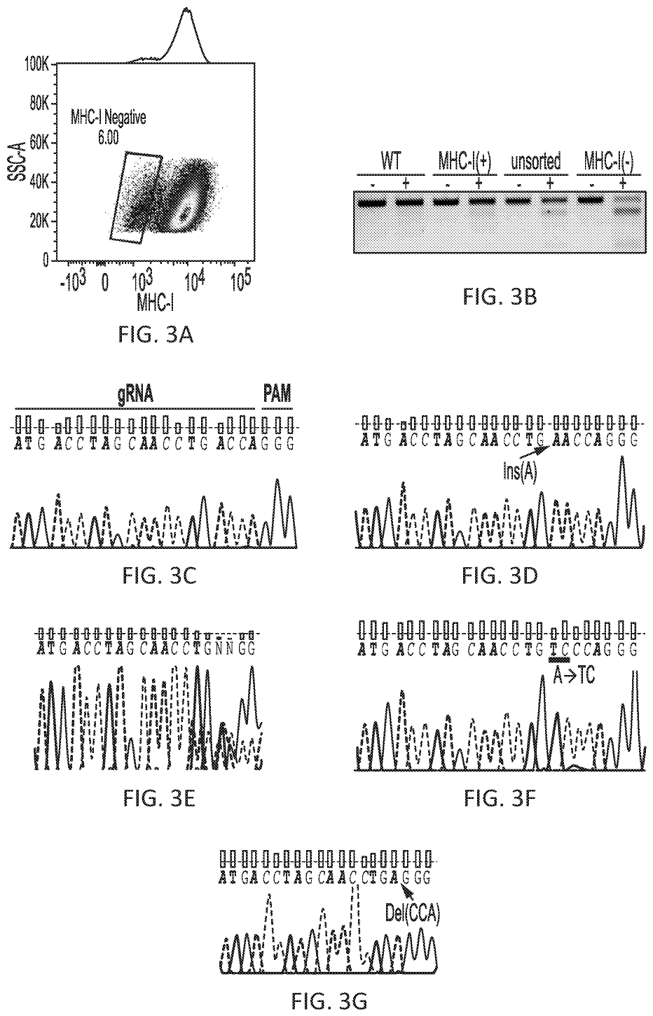

To prove that such a scheme effectively enriches for DKOs in target genes, B2M and tumor suppressor gene PTEN and B2M were targeted. MHC-I negative and positive cells were selected and sort them into single cells in isolation (FIG. 3A). Both negative and positive cells were sorted in to pools and attempted to see if sorting significantly changes the abundance of indel mutations. With ENGEN assay, it was confirmed that indel mutations in PTEN were significantly enriched in cells with negative surface MHC-I (FIG. 3B). It was then attempted to determine if DKOs were indeed highly enriched in resulting MHC-I negative single clones. Nine clones were picked each from MHC-I positive and negative colonies, and PCR amplified the targeted loci with the same primers used for ENGEN assay. The PCR products were sequenced with the PTEN reverse primer. All 9 clones from MHC-I positive cells are unaltered at this locus (FIG. 3C); on the strength of sequencing PCR products alone it was determined 3 of the 9 MHC-I negative clones were DKOs (FIG. 3D); For the remaining 6 MHC-I negative clones with illegible sequencing results, the PCR product was cloned into a plasmid vector and sequenced resulting plasmids, and were finally able to decide all 9/9 MHC-I negative colonies were true DKOs (FIG. 3E-G). The above results provide a solid proof-of-principle that FACS-based negative selection using membrane markers could be an effective method for enriching biallelic indel mutations in the target loci and greatly reduce the cost and improve the efficiency associated with CRISPR gene editing. The close to 100% DKO rate is by far unprecedented in literature. The modified cells can be used in molecular genetics studies in cell culture, and has the potential to be used in creating modified hypoimmunogenic cells for therapeutic applications, such as ESC and CAR-T cells. The close to 100% DKO rate is by far unprecedented in literature.

PTEN is a tumor suppressor gene, and as such there is a theoretical possibility that cells with PTEN double knockout gain advantage in growth and become over-represented in the tested colonies. To test if FAME is effective in other settings, indel mutations were created in two additional genes, MYC and ZMIZ1, which are oncogenes on the contrary. Expression plasmids for sgRNAs against MYC or ZMIZ1 were transfected along with sgRNA against B2M and cDNA for Cas9, and sorted for cells with positive and negative surface expression of MHC-I 5 days after transfection. Genomic DNA was then extracted from B2M positive, unsorted, and B2M negative pool of cells, and performed ENGEN assay to determine the rate of indel mutations. The results indicated that even in oncogenes MYC and ZMIZ1, FAME strategy similarly enriched the rate of indels for the genes-of-interest (FIG. 2). Oncogenes are considered pro-growth, and their knockout tends to present a disadvantage for growth. Despite that similar efficacy was achieved in enriching indels, which leads to concluding that this method can be widely applicable to most other genes, except for those truly essential ones. New sgRNAs can be designed that can effectively target B2M of other mammals. Furthermore, other suitable membrane selection marker can be used for any species, as long as they satisfy the same conditions, i.e., universal expression in all cell types, selectable with flow cytometry (with compatible antibodies), and neutral functionality.

4. Materials and Methods

Tissue culture—HEK293T cells were originally obtained from ATCC and cultured in DMEM media with 10% FBS.

Transfection—Transfection of HEK293T cells is achieved with Lipofectamine 2000 (Invitrogen, Carlsbad, Calif.) according to the instructions of the manufacturer. Tissue culture media and supplies are provided by VWR unless otherwise indicated.

Flow cytometry—Transfected HEK293T cells are stained with FITC-conjugated anti-Human HLA-A,B,C (Clone W6/32, Part number B223308 (Biolegend, San Diego, Calif.) according to protocol provided by the manufacturer, and analyzed on a Beckman Coulter Cytomics FC500 flow cytometer. The results were analyzed with Beckman Coulter CXP Software.

FACS: Staining of 293T with anti-MHC-I antibody was achieved the same way as described above in flow cytometry analysis. Sorting of MHC-I negative and positive cells is carried out in a Becton Dickinson FACSAria Ilu cell sorter in Barrow's Neurological Institute, Phoenix, Ariz.

Plasmids and cloning: pCMV-hCas9, and pENTR221-U6-sgRNA constructs were kind gifts of Dr. Branden Moriarity of University of Minnesota. The sgRNA constructs for targeting B2M, PTEN, MYC, and ZMIZ1 were created based on cloning of PCR products as described by Ran et al (Ran et al., 2013). Briefly, for each construct, PCR was conducted using diluted pENTR221-U6-sgRNA as template, common reverse primer (5′-cggtgtttcgtcctttccac-3′), and guide-specific forward primers (Table 1) to create plasmid-length PCR products, which was then treated with T4 polynucleotide kinase (New England Biolab) to enable self-ligation with T4 ligase (New England Biolab). The ligation products were used to transform competent DH10B E. coli (New England Biolab). Resulting plasmids were verified by Sanger sequencing using M13 reverse primer at DNA sequencing lab at Arizona State University (Phoenix, Ariz.). The target loci for editing of each gene by Streptococcus pyogenes Cas9 nuclease were chosen based on prediction by a cloud-based algorithm hosted at Deskgen.com.

TABLE 1
Sequence of oligos used for creating sgRNA
expression plasmids

| Target | Sequence (5′-3′) |
|---|---|
| B2M1 | CAGCCCAAGATAGTTAAGTGgttttagagctagaaatagc |
| B2M2 | ACAAAGTCACATGGTTCACAgttttagagctagaaatagc |
| B2M3 | CTGAATCTTTGGAGTACCTGgttttagagctagaaatagc |
| PTEN | ATGACCTAGCAACCTGACCAgttttagagctagaaatagc |
| MYC | CAGAGTAGTTATGGTAACTGgttttagagctagaaatagc |
| ZMIZ1 | TTGGTTACTCCCCAAACCGgttttagagctaggccaac |

*Gene specific sequence is capitalized in contrast to common plasmid sequence

ENGEN assay: The ENGEN assay was conducted with kits purchased from New England Biolabs, according to manufacturer's instruction. Briefly, PCR was conducted with provided high-fidelity polymerase for each targeted locus with primers listed in Table 2. The PCR products were denatured and reannealed for heteroduplex formation, followed by digestion with T7 endonuclease that recognizes mismatch created by mutagenesis. The digested products and undigested control were then run on 2% agarose gel to decide the presence and percentage of indel mutations as a result of CRISPR gene editing.

TABLE 2
Oligo sequences for ENGEN tests used for PTEN, MYC
and ZMIZ1

| Target | Sequence (5′-3′) | |
|---|---|---|
| PTEN* | Fwd | CCAGGCCTCTGGCTGCTGAG |
| | Rev | CGGACAATAGCCCTCAGGAAGA |
| MYC* | Fwd | CGGAGCGAATAGGGGGCTTC |
| | Rev | GGCCGGGAGTCAGCGTGAA |
| ZMIZ1 | Fwd | CAGTTGCATGACCTGTGGAC |
| | Rev | GAAGCTGGTCTTTCCAGCAG |

*from reference (Moriarity et al., 2014).

Sequencing verification of DKOs of PTEN in MHC-I negative and positive single cell colonies. Genomic DNA was extracted from expanded single cell colonies, and PCR was conducted to amplify amplicons including the targeted PTEN locus with PTEN Fwd and Rev primers (Table 2). PCR products were column purified with PCR purification kit (Thermofisher) and submitted for Sanger sequencing with PTEN rev primer (Table 2) at the DNA sequencing lab at Arizona State University. Clones with apparent wild-type sequences were identified as genetically unmodified. Those with mixed signals were further subject to cloning of the PCR product into pMini T2.0 vector with NEB PCR cloning kit (New England Biolab), unless DKO can be called unequivocally from the chromatogram based on sequencing of the PCR products. The plasmids from PCR cloning of each sample were sent for Sanger sequencing with T7 primer at Arizona State University DNA sequencing lab to confirm presence of mutations in single or both alleles.

Those skilled in the art will recognize, or be able to ascertain using no more than routine experimentation, many equivalents to the specific embodiments of the method and compositions described herein. Such equivalents are intended to be encompassed by the following claims.

### REFERENCES

Moriarity, B. S., Rahrmann, E. P., Beckmann, D. A., Conboy, C. B., Watson, A. L., Carlson, D. F., et al. (2014). Simple and efficient methods for enrichment and isolation of endonuclease modified cells. PloS One, 9(5), e96114.
Ran, F. A., Hsu, P. D., Wright, J., Agarwala, V., Scott, D. A., & Zhang, F. (2013). Genome engineering using the CRISPR-Cas9 system. Nature Protocols, 8(11), 2281-2308.
Ren, J., Liu, X., Fang, C., Jiang, S., June, C. H., & Zhao, Y. (2017). Multiplex genome editing to generate universal CAR T cells resistant to PD1 inhibition. Clinical Cancer Research: An Official Journal of the American Association for Cancer Research, 23(9), 2255-2266.
Wang, D., Quan, Y., Yan, Q., Morales, J. E., & Wetsel, R. A. (2015). Targeted disruption of the beta2-microglobulin gene minimizes the immunogenicity of human embryonic stem cells. Stem Cells Translational Medicine, 4(10), 1234-1245.
Wu, Y., Xu, K., Ren, C., Li, X., Lv, H., Han, F., et al. (2017). Enhanced CRISPR/Cas9-mediated biallelic genome targeting with dual surrogate reporter-integrated donors. FEBS Letters, 591(6), 903-913.

We claim:

1. A method comprising
a. administering CRISPR technology to a population of cells, wherein the CRISPR technology comprises one or more constructs for expressing Cas-9, sgRNA against a marker gene, and sgRNA against a target sequence;
b. performing FACS-based negative selection to establish an enriched cell population of negatively selected cells;
wherein the negatively selected cells do not comprise a marker encoded by the marker gene and do comprise a mutation in the target sequence.

2. The method of claim 1, wherein the CRISPR technology knocks out the marker gene and mutates the target sequence.

3. The method of any one of claims 1 and 2, wherein the marker gene encodes a cell surface protein.

4. The method of any one of claims 1-3, wherein the cell surface protein is not essential for cell survival.

5. The method of any one of claims 1-4, wherein the marker gene encodes β-2 microglobulin (B2M).

6. The method of any one of claims 1-5, wherein the sgRNA against a marker gene comprises the sequence CAGCCCAAGATAGTTAAGTGgttttagagctagaaatagc, ACAAAGTCACATGGTTCACAgttttagagctagaaatagc, or CTGAATCTTTGGAGTACCTGgttttagagctagaaatagc.

7. The method of any one of claims 1-6, wherein the target sequence is a nucleic acid sequence encoding PTEN, MYC or ZMIZ1.

8. The method of any one of claims 1-7, wherein the sgRNA against a target sequence comprises the sequence of ATGACCTAGCAACCTGACCAgttttagagctagaaatagc, CAGAGTAGTTATGGTAACTGgttttagagctagaaatagc, or TTGGTTACTCCCCAAACCGgttttagagctaggccaac.

9. The method of any one of claims 1-8, wherein the mutation is a biallelic indel mutation.

10. The method of any one of claims 1-9, wherein the Cas-9, sgRNA against a marker gene, and sgRNA against a target sequence are expressed from different constructs.

11. The method of any one of claims 1-10, wherein the biallelic indel mutation is confirmed

12. The method of any one of claims 1-11, further comprising, after step b), performing sequence analysis.

13. The method of claim 12, wherein the sequence analysis is Sanger sequencing.

14. The method of any one of claims 1-13, further comprising, after step b), culturing the enriched cell population.

15. The method of any one of claims 1-14, wherein the population of cells are mammalian cells.

16. The method of any one of claims 1-15, wherein the population of cells are a cell line.

17. The method of any one of claims 1-16, wherein the population of cells are cultured primary cells.

18. The method of any one of claims 1-17, wherein the population of cells are T cells.

19. The method of any one of claims 1-18, wherein FACS-based negative selection comprises administering an antibody capable of binding to the marker.

20. The method of any one of claims 1-19, wherein the antibody is an anti-MHC I antibody.

21. The method of any one of claims 1-20, wherein the antibody is an anti-B2M antibody.

22. A recombinant cell comprising one or more constructs for expressing Cas-9, sgRNA against a cell-surface marker gene, and sgRNA against a target sequence.

23. The recombinant cell of claim 22, wherein the cell-surface marker gene encodes a cell-surface protein that is not essential for cell survival.

24. The recombinant cell of any one of claims 22-23, wherein the cell-surface marker gene encodes β-2 microglobulin.

25. The recombinant cell of any one of claims 22-24, wherein the construct that expresses the sgRNA against a cell-surface marker gene comprises the sequence
CAGCCCAAGATAGTTAAGTGgttttagagctagaaatagc,
ACAAAGTCACATGGTTCACAgttttagagctagaaatagc,
or
CTGAATCTTTGGAGTACCTGgttttagagctagaaatagc.

26. The recombinant cell of any one of claims 22-25, wherein the target sequence is a nucleic acid sequence encoding PTEN, MYC or ZMIZ1.

27. The recombinant cell of any one of claims 22-26, wherein the construct that expresses the sgRNA against a target sequence comprises the sequence of
ATGACCTAGCAACCTGACCAgttttagagctagaaatagc,
CAGAGTAGTTATGGTAACTGgttttagagctagaaatagc,
or
TTGGTTACTCCCCAAACCGgttttagagctaggccaac.

28. The recombinant cell of any one of claims 22-27, wherein the cell is a T cell.

29. The recombinant cell of any one of claims 22-28, wherein the cell is a mammalian cell.

30. A nucleic acid sequence comprising three elements, wherein a first element comprises a nucleic acid sequence that encodes Cas-9, a second element comprises a nucleic acid sequence that expresses a sgRNA against a cell-surface marker gene, and a third element comprising a nucleic acid sequence that expresses a sgRNA against a target sequence.

31. The nucleic acid sequence of claim 30, wherein the nucleic acid sequence that expresses a sgRNA against a cell-surface marker gene comprises the sequence of
CAGCCCAAGATAGTTAAGTGgttttagagctagaaatagc,
ACAAAGTCACATGGTTCACAgttttagagctagaaatagc,
or
CTGAATCTTTGGAGTACCTGgttttagagctagaaatagc.

32. The nucleic acid sequence of claim 30-31, wherein the nucleic acid sequence that expresses the sgRNA against a target sequence comprises the sequence of
ATGACCTAGCAACCTGACCAgttttagagctagaaatagc,
CAGAGTAGTTATGGTAACTGgttttagagctagaaatagc,
or
TTGGTTACTCCCCAAACCGgttttagagctaggccaac.

33. A construct comprising any one of the nucleic acid sequences of claims 30-32.

34. The construct of claim 33, wherein the first element, second element and third element are operably linked.

35. A method comprising:
a. selectively knocking out a gene in a cell, wherein the gene is autosomal and encodes for a cell surface marker;
b. screening the cells of a) using FACS;
wherein said FACS identifies cells that lack the cell surface marker.

36. The method of claim 35, further comprising knocking out a gene of interest.

37. The method of any of claims 35-36, wherein the autosomal gene that encodes for a cell surface marker gene is B2M.

38. The method of any of claims 35-37, wherein the selective knock out is performed using CRISPR.

39. The method of any of claims 35-38, wherein the FACS is used to select MHC-1 negative cells.


SEQUENCE LISTING (18 sequences)

SEQ ID NO: 1 - DNA, 40 bp, Artificial Sequence
  synthetic construct; encodes sgRNA
cagcccaagatagttaagtggttttagagctagaaatagc

SEQ ID NO: 2 - DNA, 40 bp, Artificial Sequence
  synthetic construct; encodes sgRNA
acaaagtcacatggttcacagttttagagctagaaatagc

SEQ ID NO: 3 - DNA, 40 bp, Artificial Sequence
  synthetic construct; encodes sgRNA
ctgaatctttggagtacctggttttagagctagaaatagc

SEQ ID NO: 4 - DNA, 20 bp, Artificial Sequence
  synthetic construct; encodes sgRNA
cagcccaagatagttaagtg

SEQ ID NO: 5 - DNA, 20 bp, Artificial Sequence
  synthetic construct; encodes sgRNA
acaaagtcacatggttcaca

SEQ ID NO: 6 - DNA, 20 bp, Artificial Sequence
  synthetic construct; encodes sgRNA
ctgaatctttggagtacctg

SEQ ID NO: 7 - DNA, 40 bp, Artificial Sequence
  synthetic construct; encodes sgRNA
atgacctagcaacctgaccagttttagagctagaaatagc

SEQ ID NO: 8 - DNA, 40 bp, Artificial Sequence
  synthetic construct; encodes sgRNA
cagagtagttatggtaactggttttagagctagaaatagc

SEQ ID NO: 9 - DNA, 38 bp, Artificial Sequence
  synthetic construct; encodes sgRNA
ttggttactccccaaaccggttttagagctaggccaac

SEQ ID NO: 10 - DNA, 20 bp, Artificial Sequence
  synthetic construct; encodes sgRNA
atgacctagcaacctgacca

SEQ ID NO: 11 - DNA, 20 bp, Artificial Sequence
  synthetic construct; encodes sgRNA
cagagtagttatggtaactg

SEQ ID NO: 12 - DNA, 19 bp, Artificial Sequence
  synthetic construct; encodes sgRNA
ttggttactccccaaaccg

SEQ ID NO: 13 - DNA, 20 bp, Artificial Sequence
  synthetic construct; primer
ccaggcctctggctgctgag

SEQ ID NO: 14 - DNA, 22 bp, Artificial Sequence
  synthetic construct; primer
cggacaatagccctcaggaaga

SEQ ID NO: 15 - DNA, 20 bp, Artificial Sequence
  synthetic construct; primer
cggagcgaatagggggcttc

SEQ ID NO: 16 - DNA, 19 bp, Artificial Sequence
  synthetic construct; primer
ggccgggagtcagcgtgaa

SEQ ID NO: 17 - DNA, 20 bp, Artificial Sequence
  synthetic construct; primer
cagttgcatgacctgtggac

SEQ ID NO: 18 - DNA, 20 bp, Artificial Sequence
  synthetic construct; primer
gaagctggtctttccagcag